(19) United States
(12) Patent Application Publication
(10) Pub. No.: US 2004/0054288 A1
(43) Pub. Date: Mar. 18, 2004

(54) Ultrasonic apparatus with rotatable probe, surrounded by a protective resilent sleeve

(76) Inventor: Per Ehrenreich Nygaard, Soborg (DK)

Correspondence Address: Cooper & Dunham, 1185 Avenue of the Americas, New York, NY, 10036, US

(21) Appl. No.: 10/399,720

(22) PCT Filed: Oct. 11, 2001
(86) PCT No.: PCT/DK01/00664

(30) Foreign Application Priority Data
Oct. 18, 2000 (DK) BA 2000 00305

(51) Int. Cl. A61B 8/14

(52) U.S. Cl. 600/459

(57) ABSTRACT

Ultrasonic apparatus comprising an ultrasonic transducer rotatably secured to one end of a handle (3) by means of a pivot joint (5), the angular position of which can be changed, preferably stepwise. According to the invention the pivot joint (5) is surrounded by a circumferential, resilient rubber sleeve (8) closely fitting about the handle (3) and the transducer (1), respectively. As a result, the pivot joint (5) is not polluted during the use of the ultrasonic transducer (1), and accordingly the ultrasonic transducer (1) is easier to clean and disinfect/sterilize than hitherto known.

### TECHNICAL FIELD

The invention relates to an ultrasonic apparatus comprising and ultrasonic transducer rotatably secured to one end of a handle by means of a pivot joint, the angular position of which can be changed, preferably stepwise.

### BACKGROUND ART

Such ultrasonic apparatuses can for instance be used for scanning the cartoid aorta branching in the abdomen, arteries and veins in the knee area, prostate in connection with surgery as well as intraoperative scanning in general, where a high image definition is desired, and where the access is limited and difficult.

U.S. Pat. No. 4,535,781 discloses an ultrasonic apparatus, where the ultrasonic head can be rotated relative to the handle. Such a structure is encumbered with the draw-back that it is very difficult to clean and disinfect/sterilize the pivotjoint because said pivot joint comprises areas which are not easily accessible and where impurities can therefore accumulate.

### BRIEF DESCRIPTION OF THE INVENTION

The object of the invention is to provide an ultrasonic apparatus comprising a rotatably mounted ultrasonic transducer, which is compact and easier to clean and disinfect/sterilize than hitherto known.

The ultrasonic apparatus according to the invention is characterised in that the pivot joint is surrounded by a circumferential, resilient rubber member fitting closely about the handle and the transducer, respectively.

As a result, the pivot joint is not polluted during the use of the ultrasonic transducer, and accordingly it is easy to clean and disinfect/sterilize said ultrasonic transducer upon use.

Moreover, according to the invention, the resilient rubber member may be provided with one or more circumferential beads for increasing the flexibility.

The circumferential, resilient rubber member may either be glued or cast onto the handle member and/or the transducer member, whereby a particularly tight joining is obtained.

### BRIEF DESCRIPTION OF THE DRAWING

The invention is explained in greater detail below with reference to the accompanying drawings, in which

FIG. 1 illustrates an ultrasonic apparatus comprising an ultrasonic transducer rotatably secured to one end of a handle by means of a pivot joint,

FIG. 2 shows on a larger scale the pivot joint,

FIG. 3 shows an ultrasonic transducer according to the invention which is rotatably secured to one end of a handle by means of a pivot joint, said pivot joint being surrounded by a circumferential, resilient rubber member in form of a sleeve,

FIG. 4 shows on a larger scale the pivot join with the sleeve, and

FIG. 5 shows another embodiment of the pivot joint.

### BEST MODE FOR CARRYING OUT THE INVENTION

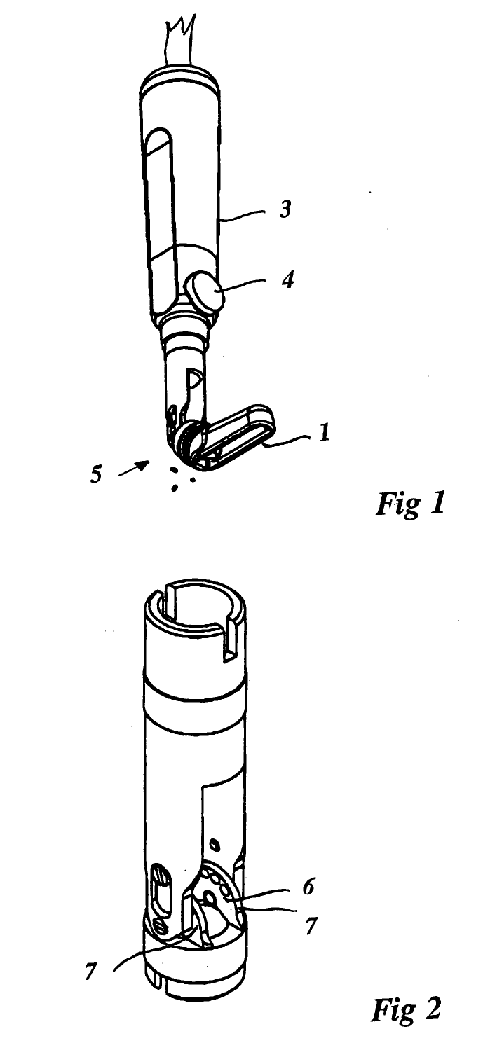

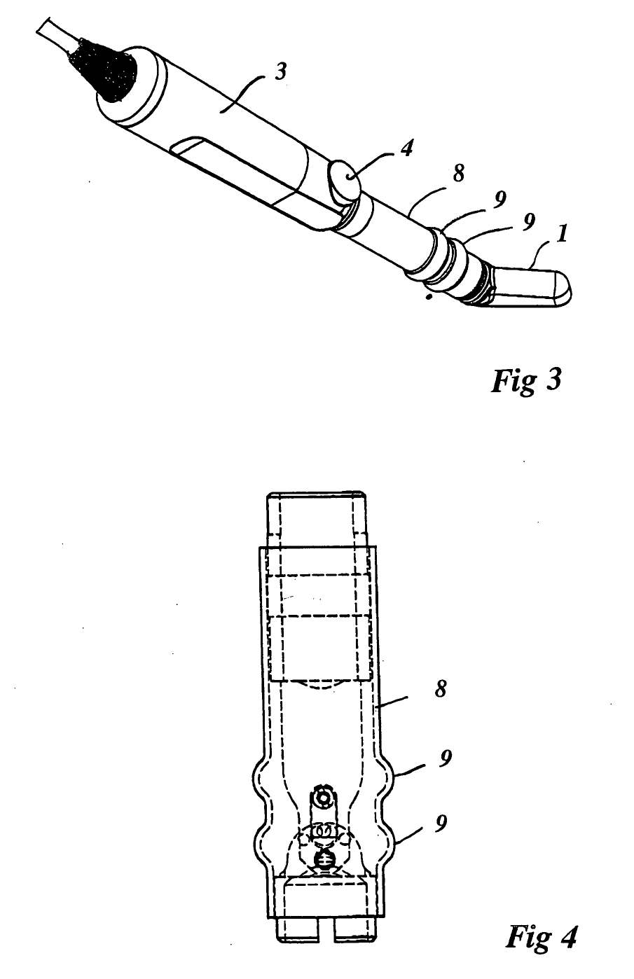

The ultrasonic apparatus shown in FIG. 1 comprises an ultrasonic transducer 1 rotatably secured to one end of a handle 3 by means of a pivot joint 5. The pivot joint 5 allows a stepwise angular adjustment for instance by means of spring biased balls. These spring biased balls can engage openings 6 along the rim of a semicircular, rotatably arranged bracket 7 fixedly connected to the transducer 1. As a result, the transducer 1 can be positioned in a desired angular position before it is used.

The ultrasonic transducer comprises a high number of transducer elements shaped and controlled in such a manner that they focus on a specific location. This capacity of the ultrasonic transducer can for instance be provided by each transducer element comprising a curved surface, optionally only in one plane. The transducer elements are connected to a high number of conductors, typically 50 to 200 conductors. As the pivot joint 5 is provided with two semicircular brackets 7 adjusting the angular position and arranged at each side of a through opening, nothing, however, prevents the large number of conductors from being extended to the transducer 1.

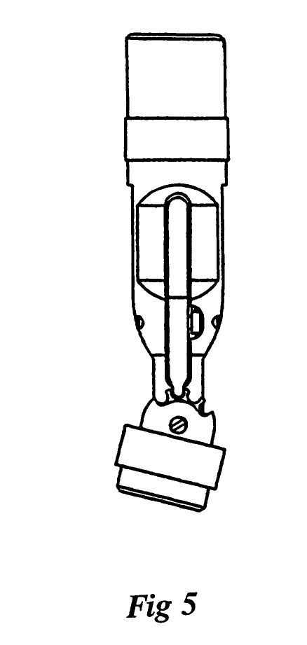

However, the pivot joint 5 can be shaped in other ways, such as by a pawl spring biased in the longitudinal direction engaging notches along the rim of the semicircular bracket, cf. FIG. 5. The portion of the pivot joint 5 connected to the handle 3 is hollow, which also applies to the portion of said pivot joint 5 being connected to the transducer 1. The handle 3 can optionally be provided with a button 4 for activating the transducer 1.

Such transducers for medical use are encumbered with a particular problem, viz. that a very careful cleaning and disinfection/sterilization is required after each use. Such a cleaning and disinfection/sterilization procedure can be very time-consuming, and if the transducer is not properly cleaned, a high risk of infection for the patients can apply.

These problems have according to the invention been solved by the pivot joint 5 being surrounded by a resilient sleeve 8 of an elastomeric material, such as rubber, said sleeve 8 closely fitting about the handle 3 and the transducer 1, respectively. The sleeve 8 is preferably glued to the handle 3 and the transducer member, respectively, in such a manner that said sleeve 8 closely fits about said members. Prior to the gluing, the relevant surfaces have preferably been roughened. The sleeve 8 can optionally be moulded onto either the handle or the transducer member, and then it fits particularly closely about said member. The sleeve 8 can optionally be provided with one or more circumferential beads 9 for increasing the flexibility of said sleeve.

The entire ultrasonic apparatus is more compact than hitherto known. The transducer member presents for instance a cross dimension of no more than 11 mm and a length of approximately 30 mm, whereas the sleeve presents an external diameter of no more than 13 mm.

In this manner an ultrasonic apparatus is provided which is very compact and which is also easy to clean and disinfect/sterilize.

1. Ultrasonic apparatus comprising an ultrasonic transducer (1) rotatably secured to one end of a handle (3) by means of a pivot joint (5), the angular position of which can be changed, preferably stepwise, characterised by the pivot joint (5) being surrounded by a circumferential, resilient member (8) of an elastomeric material, such as rubber, which closely fits about the handle (3) and the transducer, respectively, said resilient rubber member (8) being provided with one or more circumferential beads (9) for increasing the flexibility.

2. Ultrasonic apparatus as claimed in claim 1, characterised by the circumferential, resilient member (8) being glued to the handle portion (3) and/or the transducer portion.

3. Ultrasonic apparatus as claimed in claim 1, characterised by the circumferential, resilient member (8) being moulded onto the handle portion (3) and/or the transducer portion.
